(19) United States
(12) Patent Application Publication
(10) Pub. No.: US 2013/0047989 A1
(43) Pub. Date: Feb. 28, 2013

(54) METHODS AND SYSTEMS FOR ADJUSTING TIDAL VOLUME DURING VENTILATION

(71) Applicants: Joseph Doug Vandine, Manteca, CA (US); Peter Doyle, Vista, CA (US)

(72) Inventors: Joseph Doug Vandine, Manteca, CA (US); Peter Doyle, Vista, CA (US)

(73) Assignee: Nellcor Puritan Bennett LLC, Boulder, CO (US)

(21) Appl. No.: 13/223,005

(22) Filed: Aug. 31, 2011

(51) Int. Cl. A61M 16/00

(52) U.S. Cl. 128/204.23

(57) ABSTRACT

This disclosure describes systems and methods for ventilating a patient with a tidal volume that adjusts based on patient compliance. The disclosure describes a novel breath type or setting for existing breath types, that automatically and continuously varies the delivered tidal volume based on patient compliance and/or other monitored parameters.

Medical ventilator systems have long been used to provide ventilatory and supplemental oxygen support to patients. These ventilators typically comprise a source of pressurized oxygen which is fluidly connected to the patient through a conduit or tubing. As each patient may require a different ventilation strategy, modern ventilators can be customized for the particular needs of an individual patient. For example, several different ventilator modes have been created to provide better ventilation for patients in various different scenarios.

### Adjusting Tidal Volume During Ventilation

This disclosure describes systems and methods for ventilating a patient with a tidal volume that adjusts based on the patient's lung/chest wall compliance. The disclosure describes a novel breath type or setting for existing breath types, that automatically and continuously varies the delivered tidal volume based on patient compliance and/or other monitored parameters.

In part, this disclosure describes a method for ventilating a patient with a ventilator including:

a) delivering a tidal volume to a patient;

b) monitoring at least one patient parameter;

c) estimating at least one patient compliance based at least on the at least one monitored parameter;

d) adjusting the tidal volume to form an adjusted tidal volume based at least on the at least one patient compliance; and

e) delivering the adjusted tidal volume to the patient.

Yet another aspect of this disclosure describes a ventilator system that includes: a pressure generating system; a ventilation tubing system; one or more sensors; a compliance module; a tidal volume module; and a processor. The pressure generating system is adapted to generate a flow of breathing gas. The ventilation tubing system includes a patient interface for connecting the pressure generating system to a patient. The one or more sensors are operatively coupled to at least one of the pressure generating system, the patient, and the ventilation tubing system. The at least one sensor is capable of generating an output indicative of at least one monitored parameter. The compliance module estimates at least one patient compliance based at least on the output. The tidal volume module determines that a delivered tidal volume should be changed based at least on the at least one patient compliance and adjusts the tidal volume based at least on the at least one patient compliance to form an adjusted tidal volume. The processor is communicatively coupled with the pressure generating system, the one or more sensors, the compliance module, and the tidal volume module.

The disclosure further describes a computer-readable medium having computer-executable instructions for performing a method for ventilating a patient with a ventilator.

The method includes:

a) repeatedly delivering a tidal volume to a patient;

b) repeatedly monitoring at least one patient parameter;

c) repeatedly estimating at least one patient compliance based at least on the at least one monitored parameter;

d) repeatedly adjusting the tidal volume to form an adjusted tidal volume based at least on the at least one patient compliance; and

e) repeatedly delivering the adjusted tidal volume to the patient.

The disclosure also describes a ventilator system including means for delivering a tidal volume to a patient; means for monitoring at least one patient parameter; means for estimating at least one patient compliance based at least on the at least one monitored parameter; means for adjusting the tidal volume to form an adjusted tidal volume based at least on the at least one patient compliance; and means for delivering the adjusted tidal volume to the patient.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The following drawing figures, which form a part of this application, are illustrative of embodiments of systems and methods described below and are not meant to limit the scope of the invention in any manner.

FIG. 1 illustrates an embodiment of a ventilator.

FIG. 2 illustrates an embodiment of a method for ventilating a patient on a ventilator.

FIG. 3 illustrates an embodiment of a portion of the method for ventilating a patient on a ventilator as displayed in FIG. 2,

### DETAILED DESCRIPTION

Although the techniques introduced above and discussed in detail below may be implemented for a variety of medical devices, the present disclosure will discuss the implementation of these techniques in the context of a medical ventilator for use in providing ventilation support to a human patient. A person of skill in the art will understand that the technology described in the context of a medical ventilator for human patients could be adapted for use with other systems such as ventilators for non-human patients and general gas transport systems.

Medical ventilators are used to provide a breathing gas to a patient who may otherwise be unable to breathe sufficiently. In modern medical facilities, pressurized air and oxygen sources are often available from wall outlets. Accordingly, ventilators may provide pressure regulating valves (or regulators) connected to centralized sources of pressurized air and pressurized oxygen. The regulating valves function to regulate flow so that respiratory gas having a desired concentration of oxygen is supplied to the patient at desired pressures and rates. Ventilators capable of operating independently of external sources of pressurized air are also available.

While operating a ventilator, it is desirable to control the percentage of oxygen in the gas supplied by the ventilator to the patient. Further, as each patient may require a different ventilation strategy, modern ventilators can be customized for the particular needs of an individual patient. For example, several different ventilator breath types have been created to provide better ventilation for patients in various different scenarios.

For example, several ventilator breath types exist, such as volume control, pressure control, proportional assist, volume support, and pressure support breath types. These breath types utilized different measured, calculated, and/or input information to determine the how respiratory gas is delivered and removed from the patient. A previous study has shown that ventilating patients with acute lung injuries at lower tidal volumes resulted in a significantly higher rate of probability of survival when compared to ventilating these patients with acute lung injuries at higher tidal volumes. (“Ventilation with Lower Tidal Volumes as Compared with Tradition Tidal Volumes for Acute Lung Injury and the Acute Respiratory Distress Syndrome,” NEJM 2000; 342: 1301-1308.) While there is widespread recognition of the findings of this study, the use of lower tidal volumes in mechanical ventilation has not been universally adopted and, in fact, is probably not appropriate for all patients receiving mechanical ventilation.

For example, in cases where the lung has been locally or regionally injured, there are commonly areas that are damaged abutting areas that are relatively healthy. In these cases, the healthier tissue can be easily inflated with ventilation gases, while the damaged tissue remains relatively under inflated. This scenario results in sheer stress being evolved along the boundary between the two regions and causing the boundaries to become inflamed. Research has shown that the body releases endogenous cytokines, such as IL-6 and TNF-α produced in response to this local inflammation. Unfortunately, these cytokines are part of the body's defense against infection and when acting on inflamed tissues have the undesirable effect of damaging this tissue further and extending the lung injury in the lung.

Accordingly, there is a need for a ventilator mode and/or breath type that adjusts tidal volume based on changes in lung injury in the patient.

The current disclosure describes a novel breath type wherein the tidal volume (V_{T}) is indexed for patient compliance (CIV_{T} breath type). The CIV_{T} breath type relies on the presumption that there is a strong correlation between potential lung injury and low lung and/or chest wall compliance, since compliance should reduce as fluid accumulates in the tissue of the lungs from an injury or infections. Thus, the CIV_{T} breath type utilizes an algorithm that automatically sets and/or adjusts the delivered breath size according to at least the measured or estimated compliance of the lung. In some embodiments, the CIV_{T} breath type further utilizes a received patient parameter in addition to the measured or estimated compliance of the lung to automatically set and adjust the delivered breath size (or tidal volume). In some embodiments, the adjustable tidal volume of the CIV_{T} breath type is utilized as a setting instead of a breath type in addition to a volume-targeted breath type. For example, the CIV_{T} setting may be added or utilized in addition to a volume support, volume controlled, or a volume-targeted-pressure-control (VC+) breath type.

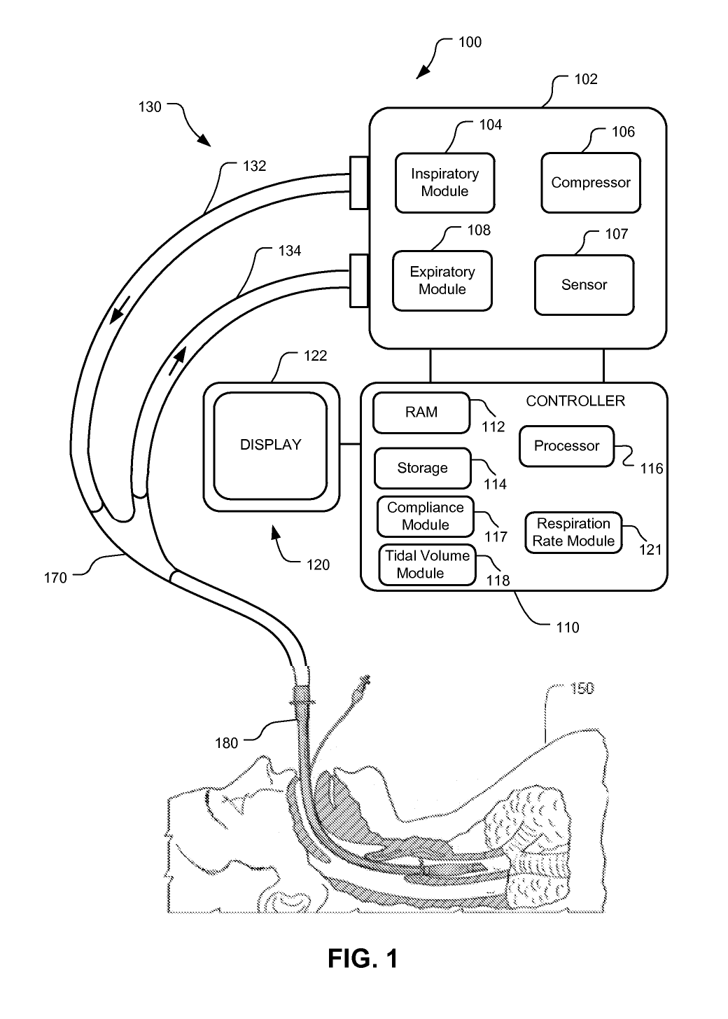

FIG. 1 is a diagram illustrating an embodiment of an exemplary ventilator 100 connected to a human patient 150. Ventilator 100 includes a pneumatic system 102 (also referred to as a pressure generating system 102) for circulating breathing gases to and from patient 150 via the ventilation tubing system 130, which couples the patient 150 to the pneumatic system 102 via an invasive (e.g., endotracheal tube, as shown) or a non-invasive (e.g., nasal mask) patient interface 180. The pneumatic system 102 generates a flow of breathing gas through the ventilation tubing system 130.

Ventilation tubing system 130 (or patient circuit 130) may be a two-limb (shown) or a one-limb circuit for carrying gases to and from the patient 150. In a two-limb embodiment, a fitting, typically referred to as a “wye-fitting” 170, may be provided to couple a patient interface 180 (as shown, an endotracheal tube) to an inspiratory limb 132 and an expiratory limb 134 of the ventilation tubing system 130.

Pneumatic system 102 may be configured in a variety of ways. In the present example, pneumatic system 102 includes an expiratory module 108 coupled with the expiratory limb 134 and an inspiratory module 104 coupled with the inspiratory limb 132. Compressor 106 or other source(s) of pressurized gases (e.g., air, oxygen, and/or helium) is coupled with inspiratory module 104 and the expiratory module 108 to provide a gas source for ventilatory support via inspiratory limb 132.

The inspiratory module 104 is configured to deliver gases to the patient 150 according to prescribed ventilatory settings. In some embodiments, inspiratory module 104 is configured to provide ventilation according to a breath type, e.g., via volume-control, pressure-control, VT, or via any other suitable breath types.

The expiratory module 108 is configured to release gases from the patient's lungs according to prescribed ventilatory settings. Specifically, expiratory module 108 is associated with and/or controls an expiratory valve for releasing gases from the patient 150.

The ventilator 100 may also include one or more sensors 107 communicatively coupled to ventilator 100. The sensors 107 may be located in the pneumatic system 102, ventilation tubing system 130, and/or on the patient 150. The embodiment of FIG. 1 illustrates a sensor 107 in pneumatic system 102.

Sensors 107 may communicate with various components of ventilator 100, e.g., pneumatic system 102, other sensors 107, processor 116, compliance module 117, tidal volume module 118, respiration rate module 121, and/or any other suitable components and/or modules. In one embodiment, sensors 107 generate output and send this output to pneumatic system 102, other sensors 107, processor 116, compliance module 117, tidal volume module 118, respiration rate module 121, and/or any other suitable components and/or modules. Sensors 107 may employ any suitable sensory or derivative technique for monitoring one or more patient parameters or ventilator parameters associated with the ventilation of a patient 150. Sensors 107 may detect changes in patient parameters indicative of patient triggering, for example. Sensors 107 may be placed in any suitable location, within the ventilatory circuitry or other devices communicatively coupled to the ventilator 100. Further, sensors 107 may be placed in any suitable internal location, such as, within the ventilatory circuitry or within components or modules of ventilator 100. For example, sensors 107 may be coupled to the inspiratory and/or expiratory modules for detecting changes in, for example, circuit pressure and/or flow. In other examples, sensors 107 may be affixed to the ventilatory tubing or may be embedded in the tubing itself. According to some embodiments, sensors 107 may be provided at or near the lungs (or diaphragm) for detecting a pressure in the lungs. Additionally or alternatively, sensors 107 may be affixed or embedded in or near wye-fitting 170 and/or patient interface 180. Indeed, any sensory device useful for monitoring changes in measurable parameters during ventilatory treatment may be employed in accordance with embodiments described herein.

As should be appreciated, with reference to the Equation of Motion for the lung, ventilatory parameters are highly interrelated and, according to embodiments, may be either directly or indirectly monitored. That is, parameters may be directly monitored by one or more sensors 107, as described above, or may be indirectly monitored or estimated by derivation according to the Equation of Motion for the lung.

The pneumatic system 102 may include a variety of other components, including mixing modules, valves, tubing, accumulators, filters, etc. Controller 110 is operatively coupled with pneumatic system 102, signal measurement and acquisition systems, and an operator interface 120 that may enable an operator to interact with the ventilator 100 (e.g., change ventilator settings, select operational modes, view monitored parameters, etc.).

In one embodiment, the operator interface 120 of the ventilator 100 includes a display 122 communicatively coupled to ventilator 100. Display 122 provides various input screens, for receiving clinician input, and various display screens, for presenting useful information to the clinician. In one embodiment, the display 122 is configured to include a graphical user interface (GUI). The GUI may be an interactive display, e.g., a touch-sensitive screen or otherwise, and may provide various windows and elements for receiving input and interface command operations. Alternatively, other suitable means of communication with the ventilator 100 may be provided, for instance by a wheel, keyboard, mouse, or other suitable interactive device. Thus, operator interface 120 may accept commands and input through display 122. Display 122 may also provide useful information in the form of various ventilatory data regarding the physical condition of a patient 150. The useful information may be derived by the ventilator 100, based on data collected by a processor 116, and the useful information may be displayed to the clinician in the form of graphs, wave representations, pie graphs, text, or other suitable forms of graphic display. For example, patient data may be displayed on the GUI and/or display 122. Additionally or alternatively, patient data may be communicated to a remote monitoring system coupled via any suitable means to the ventilator 100.

Controller 110 may include memory 112, one or more processors 116, storage 114, and/or other components of the type commonly found in command and control computing devices. Controller 110 may further include a compliance module 117, a tidal volume module 118, and/or respiration rate module 121 configured to deliver gases to the patient 150 according to prescribed breath types as illustrated in FIG. 1. In alternative embodiments, the compliance module 117, the tidal volume module 118, and/or respiration rate module 121 may be located in other components of the ventilator 100, such as the pressure generating system 102 (also known as the pneumatic system 102).

The memory 112 includes non-transitory, computer-readable storage media that stores software that is executed by the processor 116 and which controls the operation of the ventilator 100. In an embodiment, the memory 112 includes one or more solid-state storage devices such as flash memory chips. In an alternative embodiment, the memory 112 may be mass storage connected to the processor 116 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

The inspiratory module 104 receives instructions for executing a CIV_{T} breath type from the tidal volume module 118. The inspiratory module 104 delivers a tidal volume based on the received instructions from the CIV_{T} breath type to the patient 150. The tidal volume module 118 determines the tidal volume based on a measured and/or estimated patient compliance from the compliance module 117. As used herein patient compliance is a lung and/or chest wall compliance. The patient compliance may be measured or estimated dynamically, statically or at any other suitable timing. In some embodiments, the tidal volume module 118 and/or the compliance module 117 are part of the controller 110 as illustrated in FIG. 1. In other embodiments, the tidal volume module 118 and/or the compliance module 117 are part of the processor 116, pneumatic system 102, and/or a separate computing device in communication with the ventilator 100.

The compliance module 117 receives a compliance measurement or estimates compliance based on at least one monitored patient parameter from the sensor(s) 107. The compliance module receives a compliance measurement from the sensor(s) 107, from operator input, or from any other suitable component communicatively coupled to the ventilator 100. In some embodiments, the compliance module 117 estimates compliance by entering the monitored parameters into the Equation of Motion of the lung. In further embodiments, the monitored patient parameters are inspiratory flow and/or net flow. In some embodiments, the estimated compliance is based on at least one of a measured and/or estimated resistance and/or elastance.

The compliance module 117 estimates lung, and/or chest wall compliance periodically or continuously. In some embodiments, the compliance module 117 estimates compliance periodically after a predetermined amount of time has passed or at a predetermined event. For example, the compliance module 117 may estimate patient compliance every 30 milliseconds, one second, 10 seconds, 30 seconds, one minute, and/or 5 minutes. The predetermined event may include a predetermined number of breaths, such as one breath, two breaths, and/or three breaths. In other instances, the predetermined event may include the initiation or beginning of inhalation.

In some embodiments, the compliance module 117 may have the one or more sensors 107 that measure patient parameters during a short inspiratory pause before the onset of exhalation. This pause is delivered periodically before the onset of exhalation by the inspiratory module 104. The periodic pause may be delivered periodically based on a predetermined time period and/or a predetermined event. In other embodiments, the compliance module 117 utilizes measurements from one or more sensors 107 taken during ongoing inspiration and/or expiration. In other embodiments, the compliance module 117 receives a compliance measurement from the one or more sensors 107 and/or another component of the ventilator 100 and does not have to estimate or calculate compliance.

Once the compliance module 117 determines the estimated and/or measured compliance, the compliance module 117 sends and/or communicates the compliance to at least one of the processor 116, controller 110, pneumatic system 102, and/or the tidal volume module 118. If the compliance module 117 does not send the measured or estimated compliance to the tidal volume module 118 directly, another component of the ventilator 100 communicates or sends the measured or estimated compliance to the tidal volume module 118,

The tidal volume module 118 determines if the current tidal volume being delivered to the patient needs to be changed based on the received and/or measured compliance from the compliance module 117. If the tidal volume needs to be changed, the tidal volume module 118 calculates an adjusted tidal volume based at least on the estimated or measured compliance. The adjusted tidal volume is sent by the tidal volume module 118 along with instruction for executing the CIV_{T} breath type to the inspiratory module 104. As used herein, the adjusted tidal volume refers to any tidal volume determined and/or sent by the tidal volume module 118 of the ventilator 100 for delivery to the patient 150 that is different from the tidal volume previously delivered to the patient 150. In some embodiments, the adjusted tidal volume is calculated by tidal volume module 118 based on the estimated or measured compliance and on a patient parameter, such as height, weight, gender, ages, ideal body weight, and disease state. If the tidal volume does not need to be changed, the tidal volume module 118 sends instructions to the inspiratory module 104 for execution of the CIV_{T} breath type that does not adjust the previously delivered tidal volume.

In some embodiments, the adjustable tidal volume of the CIV_{T} breath type is utilized as a setting and not as a breath type. In these embodiments, the CIV_{T} setting is utilized in addition to a volume-targeted breath type. For example, the CIV_{T} setting, which adjusts the tidal volume based on the measured and/or estimated compliance may be added or utilized in addition to a volume support, volume controlled, or a volume-targeted-pressure-control (VC+) breath type. In these embodiments, the instructions sent to the inspiratory module 104 are for the execution of a volume targeted breath type that adjusts tidal volume based on a measured and/or estimated compliance.

The CIV_{T} breath type refers to a type of ventilation in which the ventilator 100 estimates potential for the extension of lung injury of the patient 150 based on the measured and/or estimated compliance. The lower the compliance, the more likely the patient 150 has lung damage. Lung infections and/or injury result in fluid accumulation in the lungs, which in turn reduces the compliance of the patient 150. In cases where the lung has been locally or regionally injured, there are commonly areas that are damaged abutting areas that are relatively healthy. In these cases, the healthier tissue can be easily inflated with ventilation gases, while the damaged tissue remains relatively under inflated. This scenario results in sheer stress being evolved along the boundary between the two regions and causing the boundaries to become inflamed. Accordingly, the CIV_{T} breath type adjusts the size of the tidal volume delivered to the patient 150 based on the measured and/or estimated lung compliance.

In some embodiments, the CIV_{T} breath type utilizes a predetermined algorithm, which incorporates the measured and/or estimated compliance to determine the amount of tidal volume to deliver to the patient 150. In some embodiments, the CIV_{T} breath type further utilizes at least one patient parameter, such as weight, height, gender, age, and/or ideal body weight in addition to the measured and/or estimated compliance to determine the proper tidal volume for the patient 150. In these embodiments, the CIV₁ breath type may utilize a predetermined algorithm, which incorporates the measured and/or estimated compliance along with at least one patient parameter to determine the amount of tidal volume to deliver to the patient 150. For example, in one embodiment, the CIV_{T} breath type will deliver an adjusted tidal volume of 4 ml/kg for a compliance measurement of 0.20 ml/cmH₂O/kg, an adjusted tidal volume of 5 ml/kg for a compliance measurement of 0.30 ml/cmH₂O/kg, an adjusted tidal volume of 6 ml/kg for a compliance measurement of 0.50 ml/cmH₂O/kg, an adjusted tidal volume of 7 ml/kg for a compliance measurement of 0.40 ml/cmH₂O/kg, an adjusted tidal volume of 8 ml/kg for a compliance measurement of 0.60 ml/cmH₂O/kg, an adjusted tidal volume of 9 ml/kg for a compliance measurement of 0.70 ml/cmH₂O/kg, and an adjusted tidal volume of 10 ml/kg for a compliance measurement of 0.80 m/kmH₂O/kg. As known by a person of skill in the art, the previous example is just one embodiment of how the ventilator 100 and/or tidal volume module 118 could index tidal volume against lung compliance. Further, the tidal volume delivered in the previous example may also be adjusted to account for different patient parameters, such as body surface area, height, weight, and/or ideal or predicted body weight.

The tidal volume module 118 determines an adjusted tidal volume and sends the adjusted tidal volume along with the instruction for executing the CIV_{T} breath type to the inspiratory module 104 periodically or continuously. In some embodiments, the tidal volume module 118 determines and/or sends the adjusted tidal volume along with the instruction for executing the CIV_{T} breath type to the inspiratory module 104 periodically after a predetermined amount of time has passed or at a predetermined event, For example, the tidal volume module 118 may determine and/or send the adjusted tidal volume along with the instruction for executing the CIV_{T} breath type to the inspiratory module 104 every 30 milliseconds, one second, 10 seconds, 30 seconds, one minute, and/or 5 minutes. The predetermined event may include a predetermined number of breaths, such as one breath, two breaths, and/or three breaths. In other instances, the predetermined event may include the initiation or beginning of inhalation,

In some embodiments, the ventilator 100 utilizes a respiration rate module 121. The respiration rate module 121 determines if the patient 150 is being non-spontaneously ventilated. During spontaneous ventilation, the patient triggers the delivery of each breath and dictates their desired respiration rate (RR). During non-spontaneous ventilation, the RR is predetermined or set by the ventilator independent of the spontaneous efforts of the patient to breath. The RR during non-spontaneous ventilation may be input by an operator and/or determined by the ventilator 100 based on patient parameters, ventilation parameters, and/or any other relevant settings and information. If the ventilator 100 is delivering spontaneous ventilation, the respiration rate module 121 continues to monitor for a switch to non-spontaneous ventilation.

If the respiration rate module 121 determines that the ventilator 100 is delivering non-spontaneous ventilation, the respiration rate module 121 will compare the delivered tidal volume to a predetermined threshold. The predetermined threshold may be an acceptable value or a range of acceptable values for the tidal volume. If the delivered tidal is equal to the predetermined threshold, the respiration rate module 121 will not change the set RR and continues to periodically or continuously to determine if the ventilator 100 is delivering non-spontaneous ventilation. If the delivered tidal is not equal to the predetermined threshold, the respiration rate module 121 will change the set RR provided by the ventilation. The respiration rate module 121 will adjust the RR proportionally in response to the delivered tidal volume to ensure that a minimum minute volume is provided to patient 150 (e.g., if tidal volume is reduced based on compliance of the patient's lungs, the RR is increased to maintain the desired minute volume and vice versa if the tidal volume is increased). Further, after the respiration rate module 121 adjusts the RR proportionally in response to the delivered tidal volume, the respiration rate module 121 will continue to periodically or continuously determine if the ventilator 100 is delivering non-spontaneous ventilation.

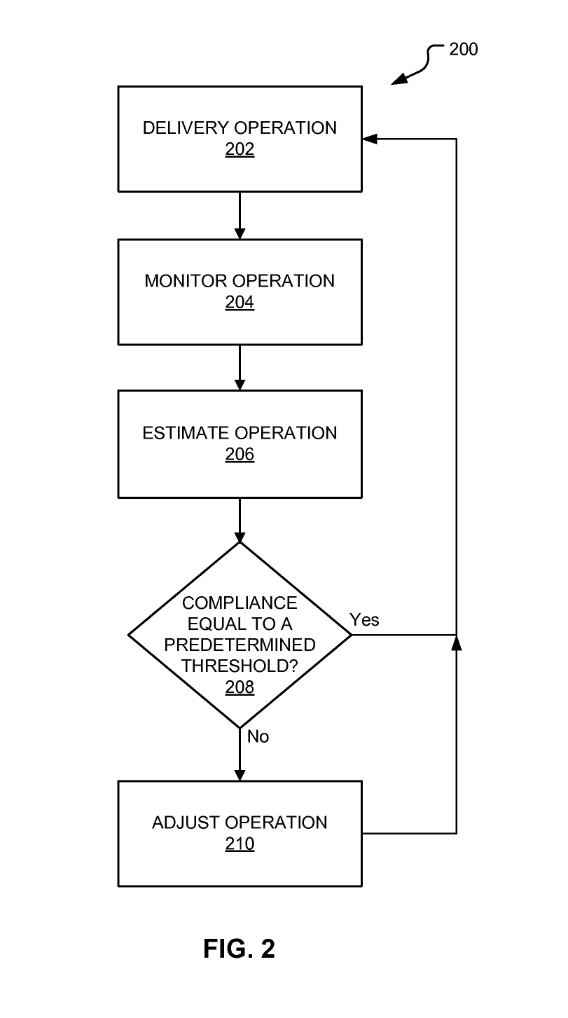

FIG. 2 illustrates an embodiment of a method 200 for ventilating a patient with a ventilator that utilizes a CIV_{T} breath type. The CIV_{T} breath type adjusts the delivered tidal volume delivered to a patient to account or compensate for a specific level of potential lung injury. In some embodiments, the adjustable tidal volume of the CIV_{T} breath type of method 200 is utilized as a setting and not as a breath type. In these embodiments, the CIV_{T} setting is utilized in addition to a volume-targeted breath type. For example, the CIV_{T} setting, which adjusts the tidal volume based on the measured and/or estimated compliance may be added or utilized in addition to a volume support, volume controlled, or a volume-targeted-pressure-control (VC+) breath type.

As illustrated, method 200 includes an ongoing delivery operation 202. During the delivery operation 202, the ventilator delivers a set tidal volume to a patient to provide ongoing respiratory therapy to the patient until such time as the settings on the ventilator are changed by the operator or automatically by this method 200. The ventilator during the delivery operation 202 may deliver an initial tidal volume or an adjusted tidal volume to the patient. The initial tidal volume delivered to the patient by the ventilator is a predetermined tidal volume delivered to begin ventilation of the patient. The predetermined tidal volume may be input by the clinician and/or determined by the ventilator. The ventilator may determine the tidal volume based on patient parameters, ventilation parameters, and/or any other suitable information. The predetermined tidal volume is delivered to the patient by the ventilator during the delivery operation 202 until the ventilator calculates an adjusted tidal volume. Once the ventilator calculates an adjusted tidal volume, the ventilator during the delivery operation 202 delivers the most recently adjusted tidal volume to the patient.

For example, the initial tidal volume and/or predetermined tidal volume may be determined by the ventilator based on body surface area, height, weight, and/or ideal or predicted body weight. In other instances, the initial tidal volume and/or predetermined tidal volume may be determined by the ventilator based on gender and/or age. In some embodiments, the predetermined tidal volume determined by the ventilator may be based on ventilator information or parameters, such as RR.

Also, method 200 includes a monitoring operation 204. During the monitoring operation 204, the ventilator monitors at least one patient parameter. In some embodiments, the patient parameters include inspiratory lung flow, net lung flow, and/or airway pressure. The monitoring operation 204 is performed by sensors. The sensors may include any suitable sensing device as known by a person of skill in the art for a ventilator. In some embodiments, the sensors are located in the pneumatic system, the breathing circuit, and/or on the patient. In some embodiments, the ventilator during monitoring operation 204 monitors the patient parameters periodically or continuously while providing ventilation to the patient at the current settings. For example, the ventilator during the monitoring operation 204 may monitor the inspiration flow every computational cycle (e.g., 2 milliseconds, 5 milliseconds, 10 milliseconds, etc.). In other embodiments, the ventilator during the monitoring operation 204 may monitor the some or all of the patient parameters as necessary for the CIV_{T} breath type every breath or after a predetermined amount of time.

In some embodiments, method 200 includes a parameter estimation operation 206. During the parameter estimation operation 206, the ventilator estimates at least one patient compliance based at least a monitored parameter. The patient compliance is lung compliance and/or chest wall compliance. The patient compliance may be measured or estimated dynamically, statically or at any other suitable timing. Further, any of these compliance measurements or estimations may or may not be corrected for the compliance of the breathing circuit. In further embodiments, the estimated lung compliance is estimated based on monitored flow and/or some algorithm such as the Equation of Motion. The estimated patient parameters may be estimated by any suitable processor found in the ventilator. In some embodiments, the estimated patient parameters are calculated by a controller, a pneumatic system, and/or a separate computing device operatively connected to the ventilator.

In some embodiments, method 200 includes a determination operation 208. During the determination operation 208, the ventilator determines that the tidal volume should be changed based at least on patient compliance estimated in the estimation operation 206. In an embodiment, the ventilator during the determination operation 208 compares the patient compliance to a predetermined threshold of patient compliance. If the ventilator determines during the determination operation 208 that the patient compliance is not equal to the predetermined compliance threshold, the ventilator selects to perform adjusting operation 210. If the ventilator determines during the determination operation 208 that the patient compliance is equal to the predetermined compliance threshold, the ventilator selects to perform delivery operation 202 to deliver the predetermined tidal volume or the most recently received adjusted tidal volume.

The predetermined compliance threshold may include a single compliance value, a range of acceptable compliance values, or an average value taken over a number of breaths. In some embodiments, the predetermined compliance value may be based on a patient parameter, such as disease state, body surface area, height, weight, age, ideal or predicted body weight, and/or gender.

Alternative methods for determining whether to adjust the tidal volume may also be used. For example, multiple criteria based on parameters others than compliance may be used in addition to or as a substitute for the compliance analysis. Such additional parameters may include lung resistance, inspiratory and expiratory flow, inspiratory pressure and patient diagnosis.

Method 200 includes an adjusting operation 210. In some embodiments, the adjusting operation 210 is performed based on the results of the determination operation 208. During the adjusting operation 210, the ventilator changes the tidal volume delivered by the ventilator to an adjusted tidal volume value that is calculated based at least on the at least one patient compliance. The adjusted tidal volume may be calculated as part of the adjusting operation 210 or may be calculated as part of an earlier operation such as the estimation operation 206. For example, if the ventilator determines that the at least one compliance is above the predetermined threshold, the ventilator during the adjusting operation 210 may increase the delivered tidal volume. In an alternative example, if the ventilator determines that the at least one compliance is below the predetermined threshold, the ventilator during the adjusting operation 210 may decrease the delivered tidal volume. In some embodiments, the amount of adjustment of the tidal volume may be further based on a patient parameter, such as disease state, body surface area, height, weight, age, ideal or predicted body weight, and/or gender.

As discussed above, the method 200 includes an ongoing delivery operation 202. The ventilator during the delivery operation 202 delivers the adjusted tidal volume to the patient as determined by the ventilator during the adjusting operation 210.

In some embodiments, the ventilator may receive an ideal body weight. For example, the ideal body weight may be entered or input by an operator. In other instances, the ideal body weight may be calculated or estimated by the ventilator based on other patient parameters, such as an input height and/or weight. The ventilator during the estimating operation 206, determination operation 208, and/or adjusting operation 210 may perform one or more of these operations based on the received ideal body weight. For example, the ventilator during the estimating operation 206 may estimate at least one compliance based at least in part on a received ideal body weight. In some examples, the predetermined threshold utilized by the ventilator during the determination operation 208 may be based at least in part on a received ideal body weight. In some examples, the ventilator may adjust the tidal volume during the adjusting operation 210 based at least in part on a received ideal body weight.

In further embodiments, the ventilator performs the estimating operation 206, determination operation 208, and/or adjusting operation 210 periodically or continuously. For instance, the ventilator may perform the estimating operation 206, determination operation 208, and/or adjusting operation 210 after a predetermined amount of time has passed or at a predetermined event. For example, the ventilator may perform the estimating operation 206, determination operation 208, and/or adjusting operation 210 every 30 milliseconds, one second, 10 seconds, 30 seconds, one minute, and/or 5 minutes. The predetermined event may include a predetermined number of breaths, such as one breath, two breaths, and/or three breaths. In other instances, the predetermined event may include the initiation or beginning of inhalation. In other embodiments, the ventilator performs the estimating operation 206, determination operation 208, and/or adjusting operation 210 in real-time or quasi-real-time.

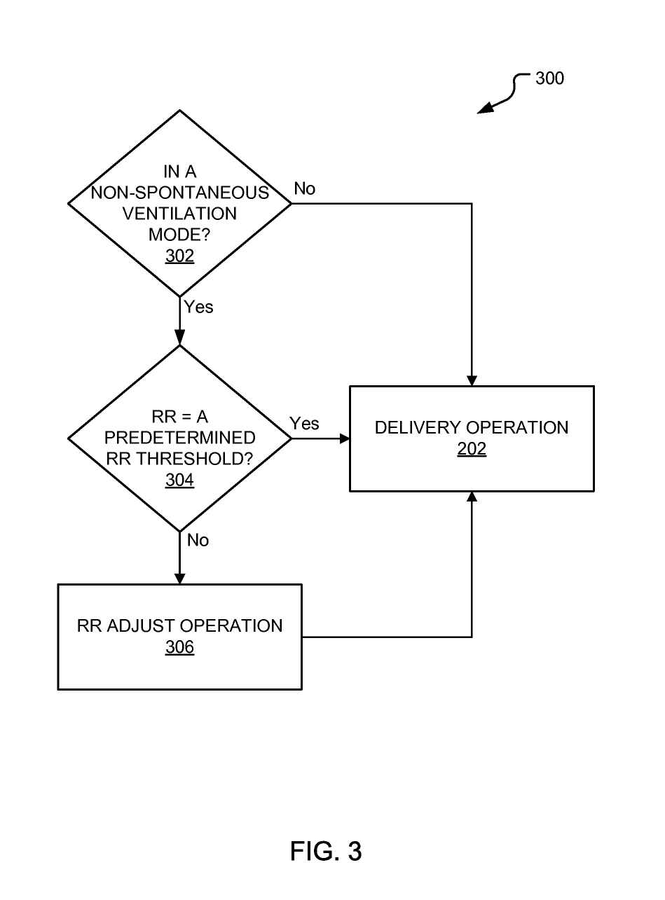

In some embodiments, delivery of a CIV_{T} breath type may further include a mode determination operation 302, a respiration rate (RR) determination operation 304, and a RR adjusting operation 306 as illustrated in FIG. 3. In an embodiment, the ventilator performs the mode determination operation 302 after the ventilator adjusts the tidal volume during the adjusting operation 210 and/or after the ventilator delivers an adjusted tidal volume during the delivery operation 202. The ventilator during the mode determination operation 302 determines if the patient is being ventilated with a non-spontaneous breath type. If the ventilator during the mode determination operation 302 determines that the patient is not being ventilated in a non-spontaneous mode (i.e., the patient is being ventilated in a spontaneous mode), then the ventilator selects to perform the delivery operation 202 as described above. The delivery operation delivers either the initial tidal volume or the most recently received tidal volume. If the ventilator during the mode determination operation 302 determines that the patient is being ventilated in a non-spontaneous mode, then the ventilator selects to perform the respiration rate (RR) determination operation 304.

In some embodiments, the ventilator changes the RR each time the tidal volume is adjusted and does so in inverse proportion. This is done to automatically maintain a desired minute ventilation. The desired minute ventilation may be input by an operator, or based on a patient parameter, such as disease state, body surface area, height, weight, age, ideal or predicted body weight, and/or gender.

The ventilator during the RR determination operation 304 determines if a RR should be changed based on the adjusted tidal volume. The ventilator during the RR determination operation 304 compares the RR to a predetermined RR threshold. If the ventilator determines during the RR determination operation 304 that the RR is not equal to the predetermined RR threshold, the ventilator selects to perform RR adjusting operation 306. If the ventilator determines during the RR determination operation 304 that the RR is equal to the predetermined RR threshold, the ventilator selects to perform delivery operation 202 to deliver the predetermined tidal volume or the most recently adjusted tidal volume. Further, the delivery operation 202 delivers the tidal volume according to the previously utilized respiration rate.

The predetermined RR threshold may include a single RR value or a range of acceptable RR values. The RR threshold may be set and/or determined to ensure that a minimum minute volume is provided to the patient. In some embodiments, the predetermined RR value may be based on a patient parameter, such as disease state, body surface area, height, weight, age, ideal or predicted body weight, and/or gender.

The ventilator during the RR adjusting operation 306 adjusts the RR based on the adjusted tidal volume to form an adjusted RR. For example, if the ventilator determines that the RR is above the predetermined threshold based on the adjusted tidal volume, the ventilator during the RR adjusting operation 306 may decrease the RR. In an alternative example, if the ventilator determines that the RR is below the predetermined threshold based on the adjusted tidal volume, the ventilator during the adjusting operation 210 may decrease the RR. In some embodiments, the amount of adjustment of the RR may be further based on a patient parameter, such as disease state, height, weight, age, ideal body weight, and/or gender.

In further embodiments, the ventilator performs the mode determination operation 302, the respiration rate (RR) determination operation 304, and/or the RR adjusting operation 306 periodically or continuously. For instance, the ventilator may perform the mode determination operation 302, the respiration rate (RR) determination operation 304, and/or the RR adjusting operation 306 after a predetermined amount of time has passed or at a predetermined event. For example, the ventilator may perform the mode determination operation 302, the respiration rate (RR) determination operation 304, and/or the RR adjusting operation 306 every 30 milliseconds, one second, 10 seconds, 30 seconds, one minute, and/or 5 minutes. The predetermined event may include a predetermined number of breaths, such as one breath, two breaths, and/or three breaths. In other instances, the predetermined event may include the initiation or beginning of inhalation. In other embodiments, the ventilator performs the mode determination operation 302, the respiration rate (RR) determination operation 304, and/or the RR adjusting operation 306 in real-time or quasi-real-time.

In further embodiments, method 200 includes a display operation (not shown). The ventilator during the display operation displays or illustrates any relevant or beneficial ventilator and/or patient information to the operator and/or patient. For example, the ventilator during the display operation may display at least one of an adjusted tidal volume, an adjusted respiration rate, the history of tidal volume adjustments over time, the estimated at least one compliance, a measured compliance, a predetermined event, and a predetermined amount of time. This list is exemplary only and is not meant to be limiting of the invention.

In some embodiments, a microprocessor-based ventilator that accesses a computer-readable medium having computer-executable instructions for performing the method of ventilating a patient with a medical ventilator is disclosed. This method includes repeatedly performing the steps disclosed in method 200 above and/or as illustrated in FIGS. 2 and 3.

In some embodiments, the ventilator system includes: means for delivering a tidal volume to a patient; means for monitoring at least one patient parameter; means for estimating at least one patient compliance based at least on the at least one monitored parameter; means for adjusting the tidal volume to form an adjusted tidal volume based at least on the at least one patient compliance; and means for delivering the adjusted tidal volume to the patient.

Those skilled in the art will recognize that the methods and systems of the present disclosure may be implemented in many manners and as such are not to be limited by the foregoing exemplary embodiments and examples. In other words, functional elements being performed by a single or multiple components, in various combinations of hardware and software or firmware, and individual functions, can be distributed among software applications at either the client or server level or both. In this regard, any number of the features of the different embodiments described herein may be combined into single or multiple embodiments, and alternate embodiments having fewer than or more than all of the features herein described are possible. Functionality may also be, in whole or in part, distributed among multiple components, in manners now known or to become known.

Thus, myriad software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software firmware components described herein as would be understood by those skilled in the art now and hereafter.

Numerous other changes may be made which will readily suggest themselves to those skilled in the art and which are encompassed in the spirit of the disclosure and as defined in the appended claims. While various embodiments have been described for purposes of this disclosure, various changes and modifications may be made which are well within the scope of the present invention. Numerous other changes may be made which will readily suggest themselves to those skilled in the art and which are encompassed in the spirit of the disclosure and as defined in the appended claims.

What is claimed is:

1. A method for ventilating a patient with a ventilator comprising:
delivering a tidal volume to a patient;
monitoring at least one patient parameter;
estimating at least one patient compliance based at least on the at least one monitored parameter;
adjusting the tidal volume to form an adjusted tidal volume based at least on the at least one patient compliance; and
delivering the adjusted tidal volume to the patient.

2. The method of claim 1, further comprising:
determining that the tidal volume should be changed based at least on the at least one patient compliance;

3. The method of claim 2, further comprising:
receiving an ideal body weight;
wherein the step of estimating, the step of determining, and the step of adjusting are further based on the received ideal body weight.

4. The method of claim 1, further comprising:
determining that the patient is being ventilated with a non-spontaneous mode;
determining that a respiration rate should be changed based on the adjusted tidal volume; and
adjusting the respiration rate based on the adjusted tidal volume to form an adjusted respiration rate.

5. The method of claim 4, wherein the step of determining the respiration rate and the step of adjusting the respiration rate are performed continuously.

6. The method of claim 4, wherein the step of determining the respiration rate and the step of adjusting the respiration rate are performed periodically based on at least one of a predetermined event and a predetermined amount of time.

7. The method of claim 6, wherein the predetermined event is a predetermined number of breaths.

8. The method of claim 1, wherein the step of estimating is performed periodically based on at least one of a predetermined event and a predetermined amount of time.

9. The method of claim 8, wherein the predetermined event is a predetermined number of breaths.

10. The method of claim 3, wherein the step of determining, the step of adjusting, and the step of delivering the adjusted tidal volume are performed periodically based on at least one of a predetermined event and a predetermined amount of time.

11. The method of claim 10, wherein the predetermined event is a predetermined number of breaths.

12. The method of claim 1, wherein the step of estimating is performed continuously.

13. The method of claim 3, wherein the step of determining, the step of adjusting, and the step of delivering the adjusted tidal volume are performed continuously.

14. The method of claim 1, wherein the at least one monitored parameter is lung flow.

15. The method of claim 1, further comprising:
displaying at least one of the adjusted tidal volume, an adjusted respiration rate, the at least one patient compliance, a predetermined event, and a predetermined amount of time.

16. The method of claim 1, wherein the ventilator is ventilating the patient with a volume targeted breath type.

17. A ventilator system comprising:
a pressure generating system adapted to generate a flow of breathing gas;
a ventilation tubing system including a patient interface for connecting the pressure generating system to a patient;
one or more sensors operatively coupled to at least one of the pressure generating system, the patient, and the ventilation tubing system, wherein at least one sensor is capable of generating an output indicative of at least one monitored parameter;
a compliance module that estimates at least one patient compliance based at least on the output;
a tidal volume module that determines that a delivered tidal volume should be changed based at least on the at least one patient compliance and adjusts the tidal volume based at least on the at least one patient compliance to form an adjusted tidal volume; and
a processor communicatively coupled with the pressure generating system, the one or more sensors, the compliance module, and the tidal volume module.

18. The ventilator system of claim 17, further comprising:
a respiration rate module that determines that the patient is being ventilated with a non-spontaneous breath mode, determines that a respiration rate should be changed based on the adjusted tidal volume, and adjusts the respiration rate based on the adjusted tidal volume to form an adjusted respiration rate.

19. The ventilator system of claim 17, wherein the compliance module determines that the delivered tidal volume should be changed based on the at least one patient compliance and on a received ideal body weight.

20. The ventilator system of claim 17, wherein the tidal volume module determines that the delivered tidal volume should be changed based on the at least one patient compliance and a received ideal body weight and adjusts the tidal volume based on the at least one patient compliance and the received ideal body weight.

21. A computer-readable medium having computer-executable instructions for performing a method of ventilating a patient with a ventilator, the method comprising:
repeatedly delivering a tidal volume to a patient;
repeatedly monitoring at least one patient parameter;
repeatedly estimating at least one patient compliance based at least on the at least one monitored parameter;
repeatedly adjusting the tidal volume to form an adjusted tidal volume based at least on the at least one patient compliance; and
repeatedly delivering the adjusted tidal volume to the patient.

22. A ventilator system, comprising:
means for delivering a tidal volume to a patient;
means for monitoring at least one patient parameter;
means for estimating at least one patient compliance based at least on the at least one monitored parameter;
means for adjusting the tidal volume to form an adjusted tidal volume based at least on the at least one patient compliance; and
means for delivering the adjusted tidal volume to the patient.
